(19) United States
(12) Patent Application Publication
(10) Pub. No.: US 2010/0003745 A1
(43) Pub. Date: Jan. 7, 2010

(54) CELL CULTURE VESSEL

(75) Inventors: Atsushi Takahashi, Kawasaki-shi (JP); Mamoru Tsukada, Fujisawa-shi (JP); Kouichi Kato, Yokohama-shi (JP)

Correspondence Address: CANON U.S.A. INC. INTELLECTUAL PROPERTY DIVISION, 15975 ALTON PARKWAY, IRVINE, CA, 92618-3731, US

(73) Assignee: CANON KABUSHIKI KAISHA, Tokyo (JP)

(21) Appl. No.: 12/493,242

(22) Filed: Jun. 29, 2009

(30) Foreign Application Priority Data
Jul. 1, 2008 (JP) 2008-172267

(51) Int. Cl. C12M 1/00

(52) U.S. Cl. 435/289.1

(57) ABSTRACT

A cell culture vessel for culturing adherent cells includes a channel allowing liquid to flow therethrough; a culture surface disposed on a wall of the channel, the culture surface allowing the adherent cells to adhere thereto; an inlet for introducing the liquid into the channel; an outlet for draining the liquid from the channel; a connecting portion communicating with the channel; and a connection receiving portion communicating with the channel. The cell culture vessel is capable of being connected to another cell culture vessel using the connecting portion or the connection receiving portion. When the connecting portion of the cell culture vessel is connected to a connection receiving portion of the other cell culture vessel, the culture surface of the cell culture vessel and a culture surface of the other cell culture vessel form a continuous culture surface.

### BACKGROUND OF THE INVENTION

1. Field of the Invention

The present invention relates to a cell culture vessel used for passage culture of adherent cells.

2. Description of the Related Art

To date, adherent cells have been mainly used for cell culture. The term “adherent cells” is a generic name for adherent-dependent cells that can survive, proliferate, and produce matter only when attached to a scaffold. Adherent cells are known to exist in many cell groups such as primary cultured cells and established cells that can indefinitely proliferate.

What is important among known characteristics of adherent cells is contact inhibition, which refers to a termination of cell proliferation that occurs when the cells have completely covered a culture surface of a culture vessel such as a dish or a flask. Another known characteristic of adherent cells is a density effect, in which deficiencies in adhesion and proliferation of the cells occur when the cells are seeded with insufficient cell density even in an environment with sufficient nutrients and oxygen. Therefore, in order to culture adherent cells that repeatedly undergo cell division, it is necessary to repeatedly perform operations for detaching the cells from a culture surface and transferring a portion of the cells to a new culture vessel every time the cells have been cultured for an appropriate period and have proliferated to a desired cell density in the culture vessel. Hereinafter, this operation is referred to as “passage”. Because passage includes very troublesome tasks such as frequent replacement of liquid, various proposals have been made in order to passage the cells with simple operations.

Japanese Patent Laid-Open No. 07-047105 describes an invention related to a splitting operation for adjusting cell density by connecting cell bags, which are made of gas-permeable resin, to each other and thereby mixing culture solutions contained in the bags.

Japanese Patent Laid-Open No. 2004-129558 proposes a method for culturing cells in a closed system by using culture vessels including a plurality of culture surfaces each having different areas. By using the method, cells are started to be cultured on a culture surface having the smallest area, and, as the proliferation of the cells progresses, the cells are detached from the culture surface with a scraper and gradually transferred to a culture surface having a larger area.

Although passage is an essential operation for performing continuous cell culture of adherent cells, passage is a very laborious task including repeatedly performing troublesome operations such as detaching, suspension, dilution, and seeding of the cells. As described above, various proposals have been made in order to simplify such troublesome passage and reduce the burden on an operator. However, a problem in that cells are inevitably affected by being detached from a culture surface has not been solved by the proposals.

In order to detach cells from a culture surface, protease such as trypsin is used. The protease digests and decomposes an adhesion factor that makes the connections between the cells and the culture medium and the connections among the cells. However, it has been pointed out that detaching of cells with trypsin or the like affects the cells (U.S. Pat. No. 5,284,766, which is a foreign patent publication corresponding to Japanese Patent Laid-Open No. 02-211865). Therefore, a method of cell culturing by which damage to the cells is reduced, that is, a method of cell culturing that does not include detaching of the cells has been desired.

Moreover, passage of the cells as a whole requires troublesome operations such as replacement of liquid using a pipette. For example, when a large number of cells are cultured using a 96-well cell culture plate, replacement of liquid is very troublesome and places a heavy burden on an operator. Furthermore, because the operations are performed in an open system, problems regarding possible infection of the operator while handling an infectious material and maintenance of a sterile environment exist.

### SUMMARY OF THE INVENTION

The invention provides a cell culture vessel that resolves the problems with cell culture, such as degradation of functions of the cells occurring when the cells are passaged and troublesome operations for passage.

The cell culture vessel includes a channel allowing liquid to flow therethrough; a culture surface disposed on a wall of the channel, the culture surface allowing the adherent cells to adhere thereto; an inlet for introducing the liquid into the channel; an outlet for draining the liquid from the channel; a connecting portion communicating with the channel; and a connection receiving portion communicating with the channel. The cell culture vessel (a first cell culture vessel) is capable of being connected to another cell culture vessel (a second cell culture vessel) using the connecting portion or the connection receiving portion. When the connecting portion of the first cell culture vessel is connected to a connection receiving portion of the other cell culture vessel, the culture surface of the first cell culture vessel and a culture surface of the second cell culture vessel form a continuous culture surface.

With the cell culture vessel, passage culture of adherent cells can be performed without carrying out a detaching operation of the cells. To be specific, because the culture surface can be extended by connecting the cell culture vessels according to the invention, passage culture of adherent cells can be performed by connecting the cell culture vessels and without carrying out a detaching operation of the cells. Thus, cell culture can be continuously performed without carrying out a detaching operation of the cells, which has been pointed out to affect cell activity but has been required in existing cell culture methods.

By attaching a liquid supply unit such as a syringe pump to the cell culture vessel, liquid can be made to flow from the inlet to the outlet. Thus, replacement of culture solution, supply of a liquid factor for inter-cell communication, and removal of waste products can be very easily carried out.

Further features of the present invention will become apparent from the following description of exemplary embodiments with reference to the attached drawings.

### BRIEF DESCRIPTION OF THE DRAWINGS

FIG. 1 is a schematic view of a cell culture vessel according to an embodiment of the invention.

FIG. 2 is a schematic view illustrating a state in which a liquid supply unit is attached to cell culture vessels structured as shown in FIG. 1.

FIG. 3 is an explanatory view illustrating an adapter used for connecting the cell culture vessels shown in FIG. 2.

FIG. 4 is a schematic view showing a state in which a liquid supply unit is attached to the cell culture vessel shown in FIG. 1 and adherent cells are being cultured in the cell culture vessel.

FIG. 5 is schematic view showing a state in which another cell culture vessel (a second cell culture vessel) is connected to the cell culture vessel shown FIG. 4 and adherent cells are being cultured in the cell culture vessels.

FIG. 6 is a schematic view showing a state in which cell culture vessels are connected to each other using an adapter including a sealing member (a screw mechanism).

FIG. 7 is a schematic view illustrating a connecting portion and a connection receiving portion of a cell culture vessel according to the invention (a configuration in which the connecting portion and the outlet are the same portion and the connection receiving portion and the inlet are the same portion, wherein a sealing member is not shown).

FIG. 8 is a perspective view of a body of the cell culture vessel according to the invention (a configuration in which a first cell culture vessel is connected to a second cell culture vessel using an adapter and the culture surface of the first cell culture vessel directly contacts the culture surface of the second cell culture vessel).

FIG. 9 is a schematic view illustrating an outline of connecting the cell culture vessels shown in FIG. 8 using the adapter.

FIG. 10 is a schematic view of an embodiment of a cell culture vessel according to the invention (a configuration in which flow of liquid to a connecting portion or a connection receiving portion is controlled using diaphragms).

### DESCRIPTION OF THE EMBODIMENTS

Hereinafter, cell culture vessels according to embodiments of the invention are described in detail with reference to the drawings. The invention is not limited to the embodiments.

### First Embodiment

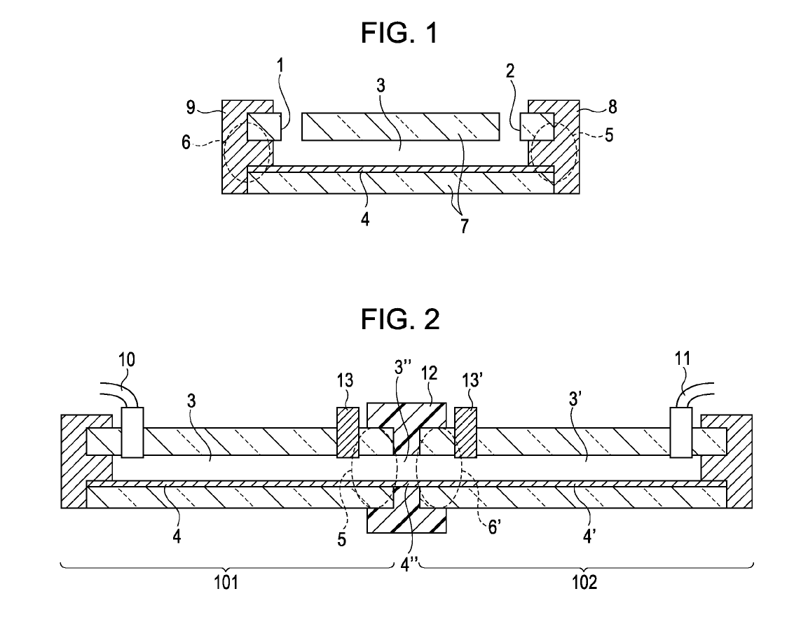

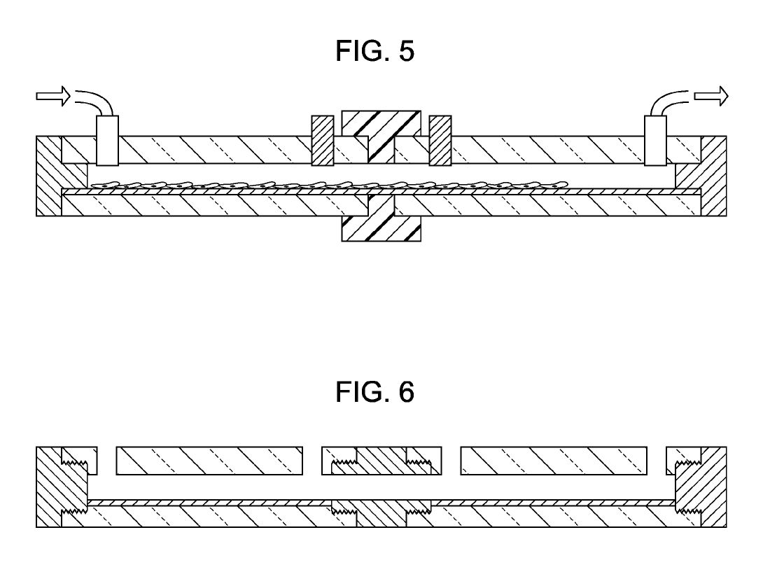

FIG. 1 is a schematic view of a cell culture vessel according to an embodiment of the invention. The cell culture vessel includes an inlet 1 for introducing liquid, an outlet 2 for draining the liquid. The inlet 1 and the outlet 2 communicate with a channel 3. As shown in FIG. 4, liquid can be supplied to the channel 3 from the inlet 1 toward the outlet 2 by using a liquid supply unit. The channel 3 is formed in a substrate 7. A culture surface 4 is formed on at least a portion of the walls of the channel 3. Adherent cells proliferate using the culture surface 4 as a scaffold. The cell culture vessel includes a connecting portion 5 and a connection receiving portion 6. The connecting portion 5 is connected to a connection receiving portion 6′ of another cell culture vessel. When the cell culture vessels are connected to each other, the culture surface 4 is connected to a culture surface 4′ (see FIG. 5) of the other cell culture vessel, so that a continuous culture surface is formed. FIG. 5 is a schematic view of a structure in which two cell culture vessels shown in FIG. 1 are connected to each other. In the example shown in FIG. 5, a connecting portion of a first cell culture vessel 101 is connected to a connection receiving portion of a second cell culture vessel 102 using an adapter 12. Cell culture vessels according to the invention may be indirectly connected to each other using an adapter or may be directly connected to each other without using an adapter.

Hereinafter, the structure of the invention is described in detail. As necessary, the term “first cell culture vessel” refers to a cell culture vessel to which another cell culture vessel is connected, and the term “second cell culture vessel” refers to the other cell culture vessel that is connected the first cell culture vessel (the cell culture vessel that is added).

### Channel

A channel is a portion through which liquid flows. Examples of the liquid include a culture solution used for proliferation of cells, a liquid factor for inter-cell communication, and cleaning liquid. The channel communicates with the inlet for introducing the liquid and the outlet for draining the liquid.

The shape of the channel is not limited to a linear shape as illustrated in the cell culture vessel shown in FIG. 1, and can be selected from among any shapes.

The channel may be formed as microscopic grooves in the substrate, or may have a capillary structure. The channel may be formed by, for example, photolithography, although the method is not limited thereto. For example, the channel can be formed by processing a substrate made of an inorganic material, such as glass, quartz glass, or silicon; or a plastic, such as PMMA (polymethylmethacrylate) or PDMS (polydimethylsiloxane), by using a method such as photolithography or molding. As necessary, a composite material may be used for the channel.

As described above, the shape of the channel may be selected from among any shapes. For example, the cross-sectional shape of the channel may be a square, a rectangle, a circle, an ellipse, etc. For example, when the cross-sectional shape of the channel is a quadrangle and the channel is formed in a flat substrate, the width of the channel can be in the range of 50 μm to 1000 μm or in the range of 20 to 100000 μm. The height of the channel can be in the range of 100 μm to 500 μm or in the range of 50 to 1000 μm. The cross-sectional shape of the channel can be uniform.

Materials used as a substrate of the cell culture vessel according to the invention are not particularly limited. For example, glass, quartz glass, silicon, or a plastic can be used. Examples of plastics that can be used as the substrate include corrosion resistant and transparent synthetic resins having specified strengths such as fluoroplastics, polycarbonate, acetal, or polystyrene.

Although the size of the substrate is not particularly limited, a substrate having a thickness in the range of 300 to 1000 μm can be used.

The substrate can be optically transparent so that the cells can be observed using an optical microscope, and can be gas-permeable so that gases such as oxygen can be easily supplied therethrough.

As necessary, surface modification may be performed on a substrate used in the invention. For example, when a glass slide, a quartz substrate, or the like is used as the substrate, a surface of the substrate can be pretreated with acid, plasma, ozone, an organic solvent, an aqueous solvent, a surface-active agent, etc. Moreover, a desired substituent can be introduced onto the surface of the substrate by treatment such as silane coupling. Furthermore, treatment for controlling surface free energy can be applied to the surface of the substrate.

### Inlet and Outlet

An inlet serves to introduce liquid into the channel, and an outlet serves to drain the liquid from the channel. By attaching a liquid supply unit, such as a syringe pump, to the inlet and the outlet, liquid can be supplied to the channel.

The inlet and the outlet can be closed as necessary. For example, in order to culture adherent cells in one cell culture vessel, a liquid supply tube is attached to the inlet 1 and a drain tube is attached to the outlet 2 of the cell culture vessel. As shown in FIG. 2, in order to culture adherent cells in two cell culture vessels that are connected to each other, a liquid supply tube 10 is attached to the inlet of the first cell culture vessel 101 and a drain tube 11 is attached to the outlet of the second cell culture vessel 102. In this case, it is necessary to close the outlet of the first cell culture vessel and the inlet of the second cell culture vessel with, for example, plugs 13 and 13′. When three or more cell culture vessels are connected to each other, both the inlets and the outlets of some of the cell culture vessels have to be closed. The method of closing the inlet and or the outlet is not particularly limited.

The inlet and the outlet may have the same shape. It is not necessary that the inlet and the outlet be strictly differentiated. The terms “inlet” and “outlet” are used only for describing the invention. The inlet and the outlet can be configured so as to be openable and closable.

### Connecting Portion and Connection Receiving Portion

As described above, a cell culture vessel according to the invention can be connected to another cell culture vessel according to the invention. The term “connecting portion” refers to a portion of a first cell culture vessel through which the first cell culture vessel is connected to the second cell culture vessel. The term “connection receiving portion” is a portion of the second cell culture vessel through which the second cell culture vessel is connected to the first cell culture vessel.

The connecting portion can be connected to the connection receiving portion directly or indirectly (using an adapter). The cell culture vessel is configured such that a continuous culture surface is formed when the cell culture vessel is connected to another cell culture vessel. The term “continuous culture surface” refers to a culture surface on which functions of the cells, such as adherence and proliferation, and activity of the cells can be continuously maintained. As long as this condition is satisfied, a surface can be regarded as a continuous culture surface even if there is a gap or a bump in an area through which the culture surface of a cell culture vessel is connected to the culture surface of another cell culture vessel. For example, a continuous culture surface may have a gap or a bump of a size equal to or less than 10 μm, which is sufficiently small as compared with the diameter of a cell body.

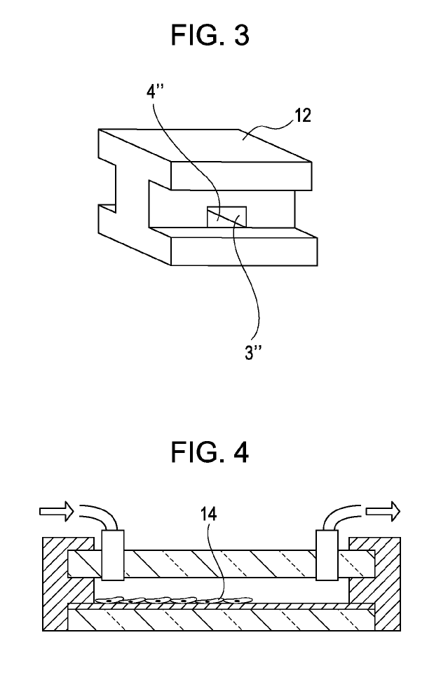

A culture surface may be formed in the channel in the adapter so that, when the connecting portion is connected to the connection receiving portion using the adapter, the culture surface of the first cell culture vessel and the culture surface of the second cell culture vessel form a continuous culture surface via the culture surface of the adapter. The disposition of the culture surface in the adapter can be deliberately designed so that a continuous culture surface is formed when the cell culture vessels are connected to each other. For example, FIG. 3 is a perspective view of an adapter used for connecting cell culture vessels structured as shown in FIG. 1. A channel 3″ is formed in the adapter, and a culture surface 4″ is disposed in the channel 3′ so that a continuous culture surface is formed when the cell culture vessels are connected to each other.

As described in detail with regard to the following embodiments, the cell culture vessel may be configured such that the inlet and the connection receiving portion are the same portion and the outlet and the connecting portion are the same portion. Likewise, the culture vessel may be configured such that the inlet and the connecting portion are the same portion and the outlet and the connection receiving portion are the same portion. In these cases, the invention can be described as follows.

The cell culture vessel includes a channel allowing liquid to flow therethrough; a culture surface disposed on a wall of the channel, the culture surface allowing the adherent cells to adhere thereto; a connecting portion disposed at one end of the channel; and a connection receiving portion disposed at the other end of the channel. The cell culture vessel (a first cell culture vessel) is capable of being connected to another cell culture vessel (a second cell culture vessel) using the connecting portion or the connection receiving portion. When the connecting portion of the first cell culture vessel is connected to a connection receiving portion of the second cell culture vessel, the culture surface of the first cell culture vessel and a culture surface of the second cell culture vessel form a continuous culture surface.

The connecting portion and the connection receiving portion can be closed by sealing members. Alternatively, the connecting portion and the connection receiving portion may include opening and closing mechanisms. However, by using removable sealing members, the inside of the cell culture vessel can be strictly separated from the external space while cells are being cultured, and the removable sealing members can be readily removed when connecting the cell culture vessels.

In order to secure close contact, a sealing agent or packing made of silicone rubber or the like can be used, as necessary, for the connecting portion, the connection receiving portion, the sealing member, and the adapter.

### Culture Surface

The culture surface is formed in the channel. When a plurality of cell culture vessels are connected to each other, the culture surfaces of the cell culture vessels are connected to each other so that a continuous culture surface is formed.

The material of the culture surface is not limited as long as cells can adhere to and be immobilized on the culture surface and that the culture surface allows observation of the cells. The culture surface can be formed as appropriate by, for example, hydrophilizing an inner surface of the channel. For example, the surface can be hydrophilized by forming an organic film or an inorganic film thereon, so that the degree of adherence of the cells to the surface can be controlled. Other examples of the method for forming the culture surface include a method of using low-temperature plasma treatment, corona treatment, ultraviolet irradiation, and a method of applying collagen, which is a protein that promotes adhesion of the cells. By masking a portion of the surface, only the remaining portion of the surface can be covered with the organic film or the inorganic film.

The shape of the culture surface may be selected from among any shapes as long as cells can adhere to and be immobilized on the culture surface. The culture surface can be a flat surface.

The culture surface can be formed over a surface of a wall of the channel. The culture surface may have any width and thickness. By appropriately determining the width and the thickness of the culture surface, the diffusion length of a liquid factor between adjacent cells can be controlled. Even in comparison with a culture vessel having a very large culture surface area, such as a culture flask, the liquid factor can be effectively delivered so that cell activity can be favorably maintained.

Adherent animal cells can be cultured in a cell culture vessel according to the invention. By using a unit configured to hold and immobilize cells on the culture surface, suspension cells can be also cultured. Examples of the method for immobilizing the suspension cells on the culture surface include a method of immobilizing an antibody that recognizes a surface antigen of a suspension cell and a method of immobilizing a known cell-anchoring material on the channel. Examples of known cell-anchoring materials include SUNBRIGHT OE-020CS (chemical name: α-succinimidyloxysuccinyl ω)-oleyloxy polyoxyethylene, made by NOF Corporation).

### Method of Culturing Cells

Referring to FIGS. 4 and 5, a method of culturing adherent cells using a cell culture vessel according to the invention is described. In the figures, the size of the cell culture apparatus and the sizes of the cells do not correspond to their actual sizes. The arrows indicate the direction in which liquid flows. Liquid such as a cell culture medium is supplied to the channel from the inlet 1 toward the outlet 2.

FIG. 4 is a schematic view showing a state in which a liquid supply unit is attached to the cell culture vessel shown in FIG. 1 and adherent cells 14 are cultured in the cell culture vessel. When the adherent cells have proliferated to the extent that the culture surface serving as a scaffold has been almost or completely exhausted, a new cell culture vessel is connected as shown in FIG. 5 so as to continue the cell culture. To be specific, the connection receiving portion 6′ of the second cell culture vessel is connected to the connecting portion 5 of the first cell culture vessel.

First, suspension liquid including the adherent cells to be cultured and a culture solution is introduced into the first cell culture vessel, and the adherent cells are cultured in the first cell culture vessel (FIG. 4). For a sealing member of the cell culture vessel, a sealing material selected from materials having excellent sealing capabilities against liquids and no reported toxicity for cells, such as silicone resin or PDMS (polydimethylsiloxane), can be used. When the cell density of the culture surface 4 has reached a desired level, the culture solution in the channel is removed, and the adapter 12 is connected to the connecting portion 5 so as to connect the second cell culture vessel. The adapter spatially connects the channel 3 of the first cell culture vessel to the channel 3′ of the second cell culture vessel, so that a continuous culture surface is formed in the first and the second culture vessels. That is, in the example shown in FIG. 5, the connection is made by using the adapter, and, because a culture surface is formed in the channel of the adapter, the culture surface 4 of the first cell culture vessel, the culture surface 4′ of the second cell culture vessel, and the culture surface 4″ of the adapter constitute a continuous culture surface. After the adapter 12 is connected to the first cell culture vessel, the connection receiving portion 6′ of the second cell culture vessel is connected to the adapter, so that the continuous culture surface is formed. Then, the cells are cultured in the connected cell culture vessels (FIG. 5). With these steps, a continuous culture surface can be extended at a desired timing. Moreover, by providing a similar connection unit to the second cell culture vessel, a new cell culture vessel can be further connected to the second cell culture vessel. Therefore, by using the cell culture vessel according to the invention, passage culture can be easily performed without carrying out a detaching operation that may affect the cells.

In order to continue passage culture from the state shown in FIG. 5, a new cell culture vessel (third cell culture vessel) can be connected to the second cell culture vessel. Whether to install the liquid supply unit or to enable opening/closing of the inlet and the outlet can be determined as appropriate. At this time, the first cell culture vessel may be removed, although the invention is not limited thereto. A sterile state can be maintained while a connection operation of cell culture vessels is being performed.

### Second Embodiment

As described above, the shapes of the connecting portion and the connection receiving portion of the cell culture vessel are not limited as long as the above-described conditions are satisfied, and can be selected from among any shapes as appropriate.

For example, a more effective sealing ability can be secured by using a screw mechanism for the sealing member, or for the connecting portion and the connection receiving portion. In the above-described embodiment of the cell culture vessel, an adapter 12 shown in FIG. 6 can be used. In order to use the screw mechanism, the culture surface can be deliberately disposed in the adapter so that a continuous culture surface is formed when the connection is made.

### Third Embodiment

As described above, a cell culture vessel according to the invention may be configured such that a connection is made without using an adapter. That is, a connecting portion and a connection receiving portion may be configured such that the connecting portion and the connection receiving portion can be directly connected to each other.

For example, the connecting portion and the connection receiving portion can be configured such that the connecting portion can be fitted into the connection receiving portion. A hook can be provided to the connecting portion or the connection receiving portion so that the connection can be released as necessary.

### Fourth Embodiment

As described above, a cell culture vessel according to the invention may be configured such that the inlet and the connection receiving portion are the same portion and the outlet and the connecting portion are the same portion. Likewise, a cell culture vessel may be configured such that the inlet and the connecting portion are the same portion and the outlet and the connection receiving portion are the same portion.

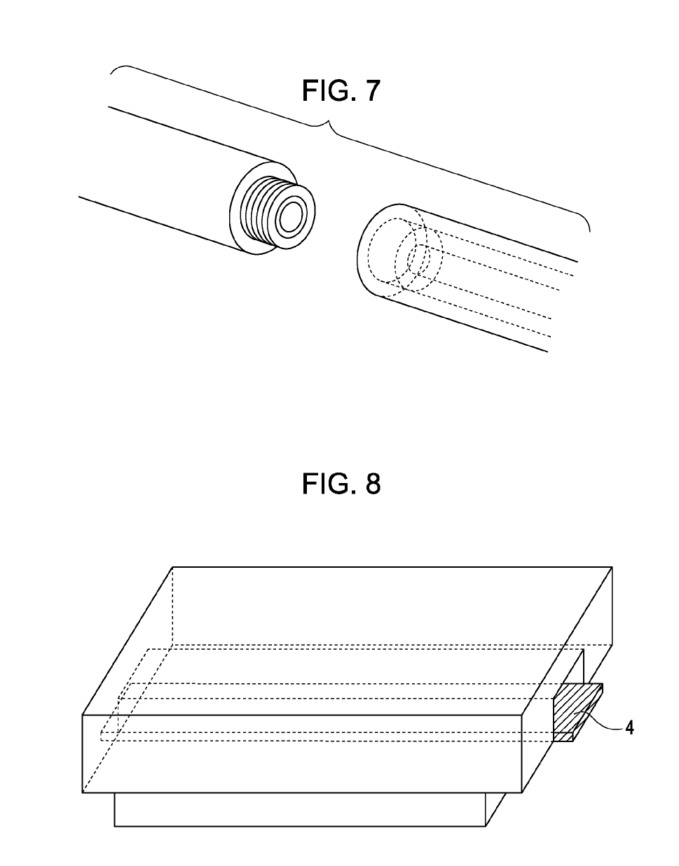

The cell culture vessel shown in FIG. 7 is an example of the cell culture vessel having this configuration. FIG. 7 is a schematic view of a connecting portion and a connection receiving portion of the cell culture vessel. The cell culture vessel is configured such that the connecting portion and the outlet are the same portion and the connection receiving portion and the inlet are the same portion, as described above.

In FIG. 7, an index line is drawn on the assumption that the connecting portion is at the right end of the cell culture vessel is and the connection receiving portion is at the left end of the cell culture vessel. Conversely, it may be assumed that the connection receiving portion is at the right end and the connecting portion is at the left end.

Likewise, in FIG. 7, it is assumed that the inlet is at the left end of the cell culture vessel and the outlet is at the right end of the cell culture vessel. Conversely, it may be assumed that the outlet is at the left end and the inlet is at the right end. These depend on how the liquid supply unit is attached to the cell culture vessel.

Although the connection mechanism is not limited to the screw mechanism, the screw mechanism is suitable for the connection mechanism because the screw mechanism allows the cell culture vessels to be easily removed and strictly sealed.

### Fifth Embodiment

A cell culture vessel according to the invention may be configured such that the cell culture vessel can be connected to another cell culture vessel using an adaptor that does not have a culture surface. That is, a first cell culture vessel is connected to a second cell culture vessel using the adapter so that a culture surface of the first cell culture vessel directly contacts a culture surface of the second cell culture vessel and thereby a continuous culture surface is formed.

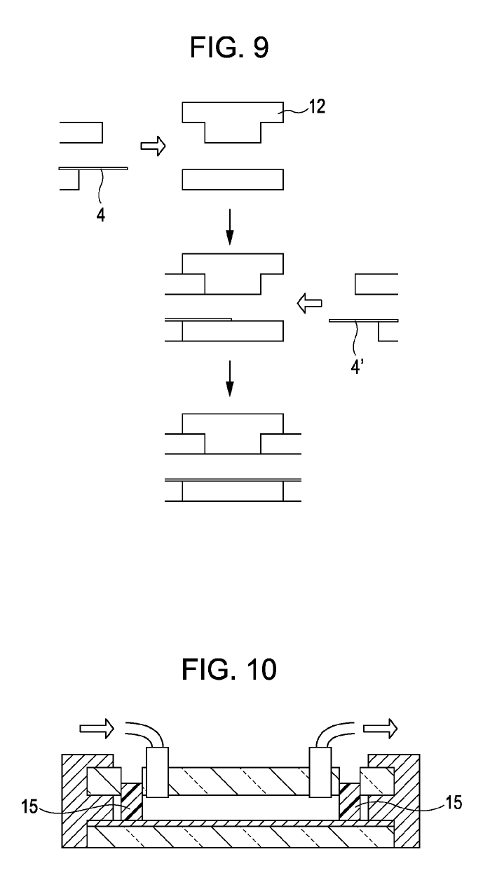

The cell culture vessel shown in FIG. 8 is an example of this embodiment. FIG. 8 is a perspective view of the body of a cell culture vessel according to the embodiment. A culture surface 4 of the cell culture vessel protrudes from the body. The cell culture vessel according to the embodiment can be connected to another cell culture vessel using an adapter 12 as shown in FIG. 9, so that a continuous culture surface is formed. In the embodiment, the adapter 12 does not have a culture surface and only serves to prevent liquid from leaking and connect the cell culture vessels.

As a modification of the embodiment, the cell culture vessel may be configured such that the flow of liquid can be controlled.

### Sixth Embodiment

A cell culture vessel according to the invention may include a liquid control unit that controls, by using a diaphragm, the flow of liquid while cells are being cultured and the flow of liquid while a connection operation of the cell culture vessels are being carried out. FIG. 10 shows an example of the cell culture vessel using the diaphragm. The arrow shows the flow of liquid. A diaphragm 15 is disposed in the channel of the cell culture vessel. The diaphragm can be driven by, for example, air pressure. A resin material such as PDMS, which has excellent sealing ability and ease of processing, can be used for the diaphragm.

First, adherent cells are cultured in a first cell culture vessel in a state in which the diaphragm is depressed (FIG. 10). A first liquid control unit and a second liquid control unit are disposed in the first cell culture vessel. At this time, liquid can be introduced and drained as shown by arrows in the figure. Next, an adapter for connecting the first cell culture vessel to the second cell culture vessel is attached to the first cell culture vessel. The adapter, which serves to connect the cell culture vessels to each other, can be made of a silicone resin such as PDMS. Then, the connection receiving portion of the second cell culture vessel is connected to the adapter, the inlet and the outlet of the channel are closed, air pressure is released, the diaphragms of the first cell culture vessel are pushed up, so that a continuous culture surface is formed between the first cell culture vessel and the second cell culture vessel. By using the culture vessel including a reversible liquid control mechanism such as the diaphragm, the culture vessels can be connected to each other without removing a culture solution. With this configuration, not only a new cell culture vessel can be added but also a cell culture vessel that has been connected can be easily detached.

### EXAMPLES

Examples of the invention are described below with reference to the drawings. However, the technical scope of the invention is not limited to the examples.

### Example 1

FIG. 8 shows a cell culture vessel used in a first example.

The method for making the cell culture vessel of the example is briefly described. First, a negative resist (SU-8; MicroChem Corp.) was applied to a silicon substrate, and a resist pattern to serve as a mold for the vessel shown in FIG. 8 was formed on the resist by photolithography. A PDMS prepolymer (Sylgard 184; Dow Corning Corp.) was poured into the mold of the resist pattern, the prepolymer in the mold was heated for one hour at temperature of 90° C. so as to form a polymer. Then, the mold was let stand in room temperature so that heat was dissipated and the PDSM was cured. Hereinafter, this process is referred to as a soft (material) lithography. After being cooled, the cured PDMS structure was detached from the mold. A slide grass to become a substrate that constitute a bottom surface was joined to the cured PDMS structure by using oxygen plasma (80 W, 30 minutes). Sealing members were attached to the connecting portion and the connection receiving portion as shown in the figure so as to prevent liquid from leaking.

Tubes for supplying liquid were fixed, using an adhesive (Shin-Etsu Silicones), to the inlet and the outlet of the PDMS structure that had been formed by photolithography as described above.

In order to pretreat the channel so that cells can adhere thereto (so that a culture surface was formed), a fibronectin/phosphate buffer (PBS) (INVITROGEN inc.) aqueous solution was introduced into the cell culture vessel through the channel, and the aqueous solution was held in a cell culture space for one hour. Then, PBS was passed through the cell culture space so as to remove excess fibronectin. HeLa cells, which are human cervical cancer cells, were suspended in a cell culture solution (RPMI1640; INVITROGEN inc.) including 10% of bovine serum (FBS; INVITROGEN inc.) so that the cell density becomes 1×10⁵ cell/ml, and the suspension was introduced into the cell culture space so that the cells were cultured in an environment of 37° C. and 5% CO₂. At this time, the cells were cultured in a static state except that a culture solution including bovine serum was passed through the vessel every six hours.

Adherence of the cells to the bottom surface of the culture vessel and proliferation of the cells at a desired speed was observed by an optical microscope. Then, the culture solution was drained from the channel, and the sealing members were removed. A PDMS structure (adapter 12) made by soft lithography was attached to the connecting portion from which the sealing member had been removed, and a new culture vessel was attached via the adapter (FIG. 9). The inside of the new culture vessel was pretreated with fibronectin so as to allow the cells to adhere thereto. A culture solution including bovine serum was introduced into the newly connected culture vessel so as to culture the HeLa cells. The HeLa cells adhered to a new cell adhesion surface (culture surface) provided by the new culture vessel and continued proliferation. It was possible not only to add a culture vessel, but also to remove a connected culture vessel, and to detach a desired one of the culture vessels. By repeating the above-described operations in accordance with an objective, it was possible to continuously perform the cell culture without carrying out a detaching operation.

### Example 2

In a second example, the culture vessel included a diaphragm that served as a valve mechanism for controlling supply of liquid. Using air pressure, the diaphragm of this example was capable of closing a channel by deforming an elastic organic film made of silicone polymer, PDMS, or the like. A PDMS sheet made as described above and stuck to the ceiling portion of the channel was used as a diaphragm sheet. The structures excluding the valve mechanism were similar to the structures in the first example. By providing the diaphragm, it was possible to detach the culture vessel without removing the culture solution from the cell culture space. The cell culture vessels were added or detached by operating the diaphragm. An example of the diaphragm using an elastic sheet is described, for example, in X. Yang, C. Grosjean, Y.-C.Tai, C.-M.Ho, Proc. IEEE MEMS '97, 114 (1997).

While the present invention has been described with reference to exemplary embodiments, it is to be understood that the invention is not limited to the disclosed exemplary embodiments. The scope of the following claims is to be accorded the broadest interpretation so as to encompass all modifications and equivalent structures and functions.

This application claims the benefit of Japanese Patent Application No. 2008-172267 filed Jul. 1, 2008, which is hereby incorporated by reference herein in its entirety.

What is claimed is:

1. A cell culture vessel for culturing adherent cells, comprising:
a channel allowing liquid to flow therethrough;
a culture surface disposed on a wall of the channel, the culture surface allowing the adherent cells to adhere thereto;
an inlet for introducing the liquid into the channel;
an outlet for draining the liquid from the channel;
a connecting portion communicating with the channel; and
a connection receiving portion communicating with the channel,
wherein the cell culture vessel is capable of being connected to another cell culture vessel using the connecting portion or the connection receiving portion, and
wherein, when the connecting portion of the cell culture vessel is connected to a connection receiving portion of the other cell culture vessel, the culture surface of the cell culture vessel and a culture surface of the other cell culture vessel form a continuous culture surface.

2. The cell culture vessel according to claim 1, further comprising:
a liquid control unit configured to control flow of the liquid to the connecting portion and the connection receiving portion,
wherein the liquid control unit includes a first liquid control unit configured to control flow of the liquid to the connecting portion and a second liquid control unit configured to control flow of the liquid to the connection receiving portion.

3. A cell culture vessel for culturing adherent cells, comprising:
a channel allowing liquid to flow therethrough;
a culture surface disposed on a wall of the channel, the culture surface allowing the adherent cells to adhere thereto;
a connecting portion disposed at one end of the channel; and
a connection receiving portion disposed at the other end of the channel,
wherein the cell culture vessel is capable of being connected to another cell culture vessel using the connecting portion or the connection receiving portion, and
wherein, when the connecting portion of the cell culture vessel is connected to a connection receiving portion of the other cell culture vessel, the culture surface of the cell culture vessel and a culture surface of the other cell culture vessel form a continuous culture surface.

4. The cell culture vessel according to claim 1,
wherein a plurality of the cell culture vessels are capable of being connected to each other, and either the culture surfaces of the cell culture vessels that are connected to each other form a continuous culture surface or the channel of the cell culture vessel and the channel of the other cell culture vessel communicate with each other.

5. The cell culture vessel according to claim 1,
wherein the connecting portion is capable of being connected to the connection receiving portion of the other cell culture vessel using an adapter or the connecting portion is capable of being directly connected to the connection receiving portion of the other cell culture vessel.

6. The cell culture vessel according to claim 1,
wherein each of the connecting portion and the connection receiving portion includes a sealing member that allows an external space and a space inside the channel to be separated from each other, the sealing member being removable.
